(19) United States
(12) Patent Application Publication
(10) Pub. No.: US 2005/0187621 A1
(43) Pub. Date: Aug. 25, 2005

(54) Foldable unitary intraocular lens

(76) Inventor: Daniel G. Brady, San Juan Capistrano, CA (US)

Correspondence Address: Advanced Medical Optics, Inc., 1700 E. St. Andrew Place, Santa Ana, CA, 92705, US

(21) Appl. No.: 10/786,894

(22) Filed: Feb. 24, 2004

(51) Int. Cl. A61F 2/16

(52) U.S. Cl. 623/6.16; 623/6.21; 623/6.49

(57) ABSTRACT

An intraocular lens has an optic made of a foldable material and at least two haptics integrally formed with the optic. The optic comprises an optical element with optical power, a transition region disposed around the entire perimeter of the optical element, and a support disposed about at least a portion of the transition region, the thickness of the support being greater than the thickness of the transition region. The transition region advantageously has a thickness of between at least about 0.07 mm and about 0.40 mm.

### BACKGROUND OF THE INVENTION

1. Field of the Invention

The present invention relates to an intraocular lens and more particularly to a foldable, unitary intraocular lens.

2. Description of the Related Art

Intraocular lenses (IOLs) are used to restore or correct vision. For example, an IOL may be placed in the anterior or posterior chamber of the human eye when cataracts or other conditions require the removal of the natural lens. Alternatively, phakic IOLs, usually implanted either in front of or behind the iris, are used to correct vision for patients still having the natural lens. IOLs typically comprise an optic for directing light toward the retina and one or more haptics for centering and stabilizing the optic within the eye.

In practice, IOLs are implanted using an insertion device through an incision in the eye. In order to reduce the size of the incision, it is common within the art to make the IOL of a foldable material such as silicone, hydrogel, acrylic, or some hybrid/combination of the same. Thus, the optic is deformable enough to be rolled or folded during the insertion procedure through an incision that is smaller than the undeformed diameter of the optic. Among other advantages, use of a small incision generally mitigates patient trauma and reduces the healing time.

In certain instances, IOLs are fabricated using a multi-piece design in which the optic and the haptics elements are made from different materials. For example, the lens may be made of one of the foldable materials listed above, while the haptics are made of a rigid, non-deformable material such as polypropylene or polymethylmethacrylate (PMMA) in the form of fine, hair-like strands. One problem with this approach is the difficulty in attaching the haptic strands to the optic in a way that will assure that the strands will not pull out from the deformable optic. The stiffness of these haptics may also present a problem during insertion and placement of the IOL within the eye. It has also been observed (e.g., U.S. Pat. No. 5,716,403—Tran et al., incorporated by reference as if fully set forth herein) that the centration force exerted by haptics made of materials such as polypropylene or PMMA tends to decay over time. This can result in poor centration of the IOL over time or the use of undesirably high initial haptic stiffness in order to compensate for the decay over time.

For these and other reasons, unitary or one-piece IOLs, in which the lens and haptics are integrally formed from a single material, may offer certain advantages over multi-piece IOLs. For example, unitary IOLs may offer advantages over multi-piece IOLs in terms of attachment, manufacturing, and mechanical performance after implantation. However, in order to avoid flexing the IOLs optic, which can degrade the optical performance, and to maintain proper positioning of the IOL within the eye, unitary IOLs made of foldable materials generally have relatively thick optic and haptic elements in comparison to multi-piece versions. The thicker IOL elements increase the size of the folded IOL, leading to an undesirable increase in the size of the incision made in the eye for insertion of the IOL.

Unitary IOLs, along with methods of fabrication and use thereof, are desired that will allow smaller ocular incisions to be used than are used for insertion of existing unitary IOLs. One method of achieving smaller incision sizes is to provide a foldable, unitary IOL having a reduced thickness relative to a prior art lens of similar optical performance. By reducing the thickness of the optic, the resulting IOL may be rolled or folded in a way that favorably decreases the size of the incision necessary to insert the lens into a subject's eye. The present invention likewise mitigates other longstanding needs by having better consistency in terms of emplacement, centration, and general optic performance.

### SUMMARY OF THE INVENTION

The present invention provides a unitary IOL that may be advantageously folded so as to allow a smaller ocular incision to be used than is currently possible using prior art unitary IOLs. The IOL provides an optic with a support that isolates an optical element in the center of the optic from forces that are produced by at least two haptics attached to the support when the IOL is implanted into a subject's eye. Isolation from these haptic forces advantageously allows the optical element of an IOL according to embodiments of the present invention to be made very thin, since the optical element is less prone to bending and deformation induced by haptic forces. The thinner optical element allows the IOL to be more tightly folded than an equivalent IOL having a thicker optical element, thus allowing a smaller incision to be used during insertion and reducing patient trauma and healing time.

One aspect of the invention involves an intraocular lens that comprises an optic made of a foldable material and at least two haptics integrally formed with the optic. The optic has an optical element with optical power, a transition region disposed around the entire perimeter of the optical element, and a support disposed about at least a portion of the transition region. The transition region disposed has a thickness of between at least about 0.07 mm and about 0.40 mm. The thickness of the support is greater than the thickness of the transition region. The haptics are coupled to the support.

The intraocular lens may be made of a material selected from a group of deformable materials consisting of hydrogel, silicone, acrylic, and hybrid combinations of the same. The optic may include a peripheral edge configured to inhibit cell growth on the intraocular lens. The intraocular lens is adapted for insertion into the eye of a human or animal subject and may be disposed, for example, in the capsular bag, anterior chamber, or posterior chamber of the eye.

The support may be disposed around the entire perimeter of the transition region or may be disposed at specific locations about the perimeter of the transition region. Each haptic may be attached to only one location on the support or, alternatively, to at least two locations on the support. The haptics of the intraocular lens may a substantially planar surface and may additionally comprise a pair of pincer arms. The pincer arms may be used, for example, to attach the intraocular lens to the iris of the eye.

The support generally has a thickness of between at least about 0.25 mm and about 0.60 mm, while the thickness of the transition region is generally at least about 0.12 mm. In certain configurations, the thickness of the haptics is advantageously less than or equal to the thickness of the support.

In another aspect of the invention, an intraocular lens comprises an optic made of a foldable material, at least two haptics integrally formed with the optic, and means for isolating the positioning force from the optical element and the transition region. In such embodiments, the optic comprises an optical element with optical power and a transition region around the entire perimeter of the optical element, the transition region having a thickness of between at least about 0.07 mm and about 0.40 mm. The at least two haptics may be adapted to produce a positioning force when inserted into an eye.

In yet another aspect of the invention, a method of manufacturing an intraocular lens comprises providing a foldable material and forming the material to produce an optic having an optical element, a transition region, and a support. The method further comprises forming the material to produce at least two haptics, the thickness of the haptics being less than or equal to the thickness of the support.

In still another aspect of the invention, a method of inserting an intraocular lens into an eye comprises providing an intraocular lens according to embodiments of the present invention and folding the intraocular lens for insertion into the eye of a subject. The insertion method further comprises creating an incision in the eye and inserting the intraocular lens through the incision and into a portion of the eye.

### BRIEF DESCRIPTION OF THE DRAWINGS

Embodiments of the present invention may be better understood from the following detailed description when read in conjunction with the accompanying drawings. Such embodiments, which are for illustrative purposes only, depict the novel and non-obvious aspects of the invention. The drawings include the following eight figures, with like numerals indicating like parts:

FIG. 1 is a front view of an IOL made according to an embodiment of the present invention.

FIG. 2 is a sectional view of the IOL shown in FIG. 1 taken generally along Line 2-2.

FIG. 3 is an enlarged view of the IOL shown in FIG. 1 schematically illustrating thicknesses of a haptic, transition region, and support of the IOL.

FIG. 4 is a front view of an IOL made according to another embodiment of the present invention.

FIG. 5 is a front view of an IOL made according to an alternate embodiment of the present invention.

FIG. 6 is a front view of an IOL made according to a further embodiment of the present invention.

FIG. 7 is a front view of an IOL made according to an additional embodiment of the present invention.

FIG. 8 is a front view of an IOL made according to yet still a further embodiment of the present invention.

### DETAILED DESCRIPTION

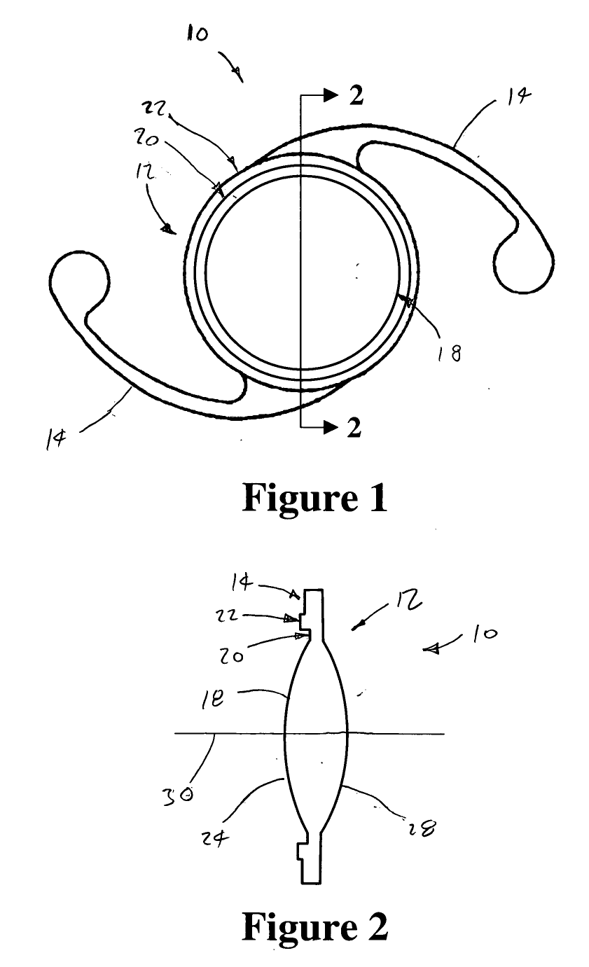

In one embodiment of the present invention, illustrated in FIGS. 1-3, an intraocular lens (IOL) 10 comprises an optic 12 made of a foldable material and at least two haptic members 14 integrally formed with the optic 12. The optic 12 comprises an optical element 18 with optical power and a transition region 20 around the entire perimeter of the optical element 18, the transition region 20 having a thickness tₑ of ranging from approximately 0.07 mm and about 0.40 mm. The optic 12 further comprises a support 22 disposed about at least a portion of the transition region 20, the thickness tₛ of the support 22 being greater than the thickness tₑ of the transition region 20. The haptics 14 are coupled to the support 22. As used herein and applied to the IOL 10, the term “integrally formed” is used to mean that the optic 12 and the haptics 14 are formed as a single piece having a substantially homogeneous material composition throughout.

The IOL 10 may be made of any foldable material allowing at least some amount of bending or flexing of the IOL 10, including materials currently used in the art (e.g., silicone, hydrogel, or acrylic) or other materials that may be developed or otherwise found to provide desirable IOL optical and mechanical properties. For example, it is anticipated that the IOL 10 could be made of hybrid materials combining silicone and acrylic to provide improved optical and/or mechanical characteristics.

The foldable material may be selected to be compatible for use with an insertion tool for delivering the IOL 10 to the eye of a subject. For instance, the foldable material may be selected to withstand the forces produced by the insertion tool so that the optical element 18 maintains good optical quality after insertion into the eye by returning to the same shape as it had prior to being loaded into the insertion tool. The foldable material of the IOL 10 may also comprise other constituents or additives in at least a portion of the IOL 10 that to enhance the performance of that portion. For example, the IOL 10 in the region of the optic 12 may contain a constituent to attenuate the transmission of radiation in the ultraviolet, infrared, or some portion of the visible waveband, such as in the violet or blue wavebands.

As illustrated in FIG. 2, the optical element 18 has an anterior side 24, a posterior side 28, and an optical axis 30 passing through the centers of the anterior and posterior surfaces of the optical element 18. The optical element 18 of the optic 12 is adapted to transmit light incident on an eye into which the IOL 10 is to be implanted and to direct that light onto the retina. As used herein, the term “eye” generally refers to a human eye; however, embodiments of the invention, such as the IOL 10, may also be adapted for use in animal subjects, either for the purpose of vision correction or for experimentation in the development of IOLs to be used in human subjects.

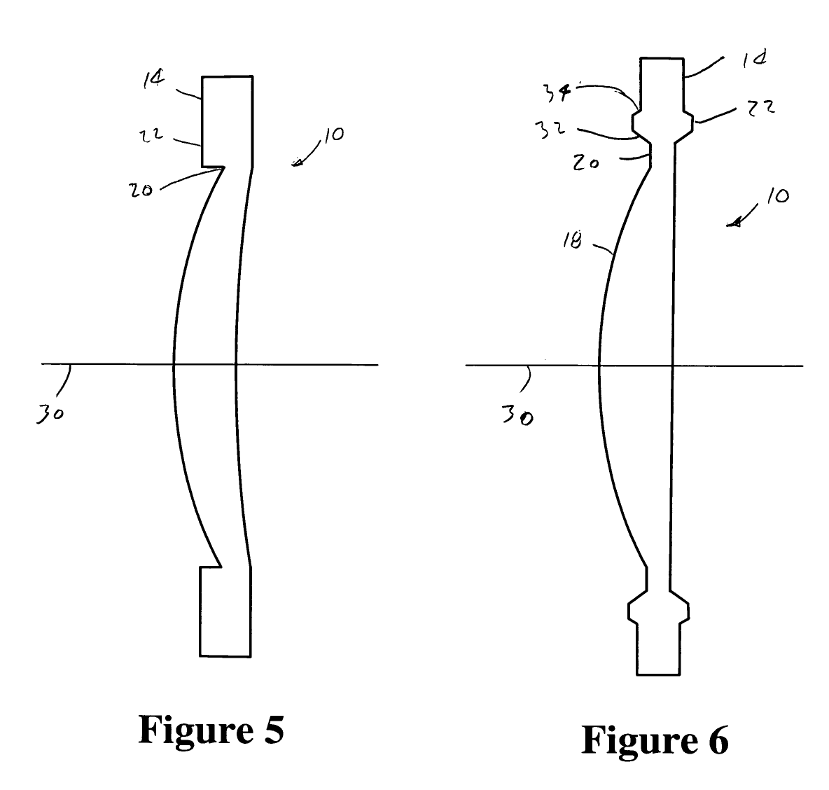

The optical element 18 may comprise any type of optical device for providing optical power or for otherwise conditioning incident light for the purpose of enhancing the vision of the eye. For example, as illustrated in FIG. 2, the surfaces on the anterior and posterior sides 24, 28 of the optical element 18 may each comprise a convex surface to provide a positive Diopter power. Alternatively, the posterior side 28 of the optical element 18 may be convex and to form a meniscus lens (e.g., FIG. 5) or substantially planar to provide a plano-convex with a positive Diopter power (e.g., FIG. 6). In other embodiments, the surfaces of the optical element 18 on the anterior and posterior sides 24, 28 may be formed to provide a negative Diopter power. Other surface configurations for the optical element 18 are also consistent with embodiments of the IOL 10, including but not limited to aspheric surfaces, multi-focal configurations, and/or the use of diffractive gratings or elements.

The support 22 is disposed about at least a portion of the perimeter of the optic 12 and is used, at least in part, for isolating the optical element 18 and the transition region 20 of the optic 12 from positioning forces produced by the haptics 14 when the IOL 10 is positioned in an eye. Such positioning forces may be produced, for instance, when edges of the haptics 14 push against the sides of the capsular bag to center the IOL 10 within the eye or, alternatively, when the haptics 14 are used to attach the IOL 10 to the iris of the eye. The thickness tₛ of the support 22 is preferably between at least about 0.25 mm and about 0.60 mm. The term thickness, as used herein and applied to the various elements of the IOL 10, refers the dimension generally along a line parallel to the optical axis 30, unless otherwise specified.

In certain embodiments, the isolation of the optical element 18 and the transition region 20 provided by the support 22 is a result of its thickness tₛ, where the thickness tₛ of the support 22 provides a region of relatively higher stiffness or rigidity compared to the thinner of the transition region 20. The thickness tₛ of the support 22 may also be greater than the thickness tₕ of the haptics 14 in the region where the haptics 14 are coupled to the support 22; however, the thickness of the support 22 may alternatively be substantially equal to the thickness of the haptics 14, as illustrated in FIG. 5. In other embodiments, the thickness of the support 22 may be greater than or equal to the thickness of the haptic 14 in the region where the haptics 14 are coupled to the support 22, but other portions of the haptic 14 are thicker than the thickness of the support 22, as dictated by the parameters for a particular haptic design.

In certain embodiments, the isolation afforded by the support 22 is not provided by the thickness and the support 22, but rather is provided by an inlay (not shown) that is stiffer than the material used to fabricate the inner portions of the IOL 10. In yet other embodiments, the stiffness of the support 22 is enhanced by selectively processing the material specifically in the region of the support 22, for instance by treating the support 22 or by impregnating the support 22 with a different substance. Using such selective processing the support 22, the optical element 18 is again isolated by the support 22 from positioning forces produced by the haptics 14 when the IOL 10 is positioned into an eye.

In certain embodiments, the support 22 is disposed around substantially the entire perimeter of the transition region 20, as illustrated in FIG. 1. In other embodiments, the support 22 is disposed only about a portion of the perimeter of the transition region 20, as illustrated in FIG. 4. In either case, the haptics 14 are coupled to the support 22 in a manner that isolates the forces produced by the haptics 14 from the optical element 18 and the transition region 20 of the optic 12.

Since the support 22 isolates edge element 20 and the optical element 18 from the haptics 14, the optical element 18 has less of a tendency to deform or bend due to forces produced by the haptics 14 when the IOL 10 is placed in the eye. As a result, the overall thickness of the optical element 18 along the optical axis 30 can be less than that of a comparable prior art IOL having substantially the same optical characteristics and aperture size. In certain instances, the thickness of the optical element 18 is determined, at least in part, by the thickness of the transition region 20 and the curvature of the surfaces of the optical element 18. The thickness of the transition region 20, in turn, may be determined by fabrication methods, the mechanical properties of the foldable IOL material, the degree of isolation provided by the support 22, and the amount of deformation or bending of the optical element 18 that can be tolerated. Based on present material and fabrication capabilities in the art, the transition region has a thickness tₑ that is preferably between at least about 0.07 mm and about 0.40 mm, and even more preferably at least about 0.12 mm. It is anticipated that the thickness tₑ of the transition region 20 may be further reduced as materials with more favorable mechanical properties are developed.

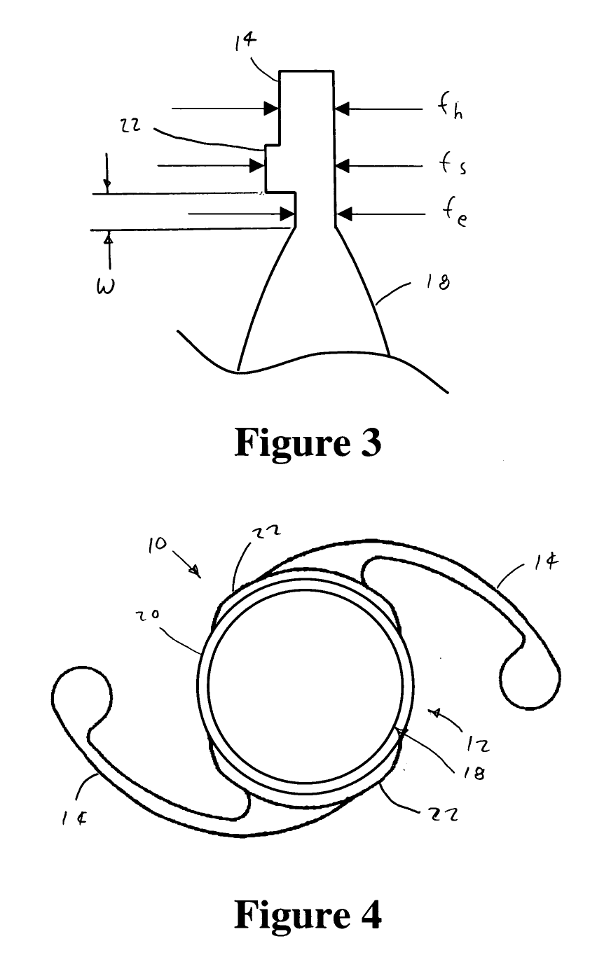

As illustrated in FIG. 1, the support 22 may protrude from the surface of the transition region 20 towards the anterior side 24 of the IOL 10. This geometry may, in certain situations, provide favorable vaulting characteristics once the IOL 10 is disposed in the eye. Alternatively, the support 22 may protrude from the surface of the transition region 20 towards the posterior side 28 of the IOL 10 or from both sides of the transition region 20, as illustrated in FIG. 6. The support 22 may be configured with relatively sharp corners as illustrated in FIG. 3; however, the corner may also be rounded or absent altogether so that one or more of the transitions between the haptics 14, the support 22, and/or the transition region 20 is smooth and less clearly defined than the sharp transitions shown in FIGS. 1 and 2. For instance, as illustrated in FIG. 6, the support 22 may comprise a beveled portion 32 between the support 22 and the transition region 20, and a beveled portion 34 between the support 22 and the haptic 14.

By reducing the thickness of the optical element 18, the IOL 10 may be rolled or folded in a more compact fashion for insertion into the eye through a very small incision. Using embodiments of the IOL 10, the incision in the eye has a dimension that is preferably less than about 2.8 mm and even more preferably less than about 2.5 mm. Such a reduced incision size is achieved without substantial detriment, for example, to the optical properties or power of the optical element 18 of the IOL 10. In the case of a cataract surgery, this may preclude the need for an incision that is larger than that used during the phacoemulsification procedure for removal of the natural lens.

As illustrated in FIG. 3, the transition region 20 has a width w and may, in certain embodiments, be used to further isolate the optical element 18 from forces produced by the haptics 14 subsequent to placement of the IOL in the eye. The width of the transition region 20 may be substantially zero or may be defined in terms of the size of a small radius between the optical element 18 and the support 22 to provide a smooth transition between those two components. The transition region 20 may also be used to reduce or prevent glare by allowing the overall diameter of the optic 12 to be increased by an amount sufficient to reduce or preclude light interacting with the periphery of the optic 12 when the pupil of the eye is fully or partially dilated. The transition region 20 may also be rounded, roughened, or otherwise formed to reduce glare from light incident on the transition region 20 or the adjacent optical element 18 and/or support 22. By roughening one or both surfaces of the transition region 20, glare is reduced as a result of light scattering by the roughened surface(s). Alternatively or additionally, the amount of glare can be attenuated by reducing the overall transmission of the transition region 20 and/or support 22. This may be accomplished by impregnating or covering the surfaces of the transition region 20 and/or support 22 with a substance having reduced transmissivity to light.

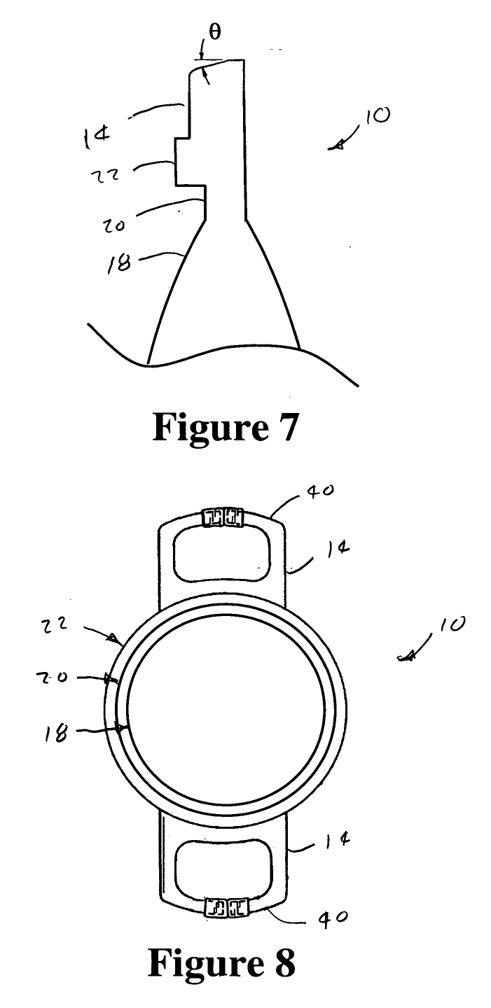

The peripheral edges of the IOL 10 can also be geometrically configured to reduce the amount of glare produced as light enters the eye. Such edge designs can also incorporate features and structures so as to inhibit cell growth on the intraocular lens 10. One example of an edge design for reducing glare and inhibiting cell growth on the IOL 10 is illustrated in FIG. 7. Other examples of such design features in IOLs that may also utilize embodiments of the present invention are taught in U.S. Pat. No. 6,468,306 (Paul et al.), which is herein incorporated by reference.

The haptics 14 may be any of the various types or designs used in the art. In certain embodiments, each haptic 14 attaches at least at one location on the support, as illustrated in FIG. 1. In other embodiments, each haptic 14 attach at least at two locations on the support 22, for instance when each of the haptics 14 forms a closed loop. In yet other embodiments, as illustrated in FIG. 8, each haptic 14 forms a substantially planar surface and/or comprises a pair of pincer arms 40. U.S. Pat. No. 6,409,763 (Brady), which is herein incorporated by reference, teaches an iris-supported IOL that incorporates this type of haptic. In other embodiments, each of the haptics 14 may have a different structure, as dictated by a particular design.

The IOL 10 may be adapted for placement in either the capsular bag or the anterior or posterior chambers of the eye. For instance, the embodiment of the IOL 10 illustrated in FIG. 8 is particularly suited for placement in the anterior chamber of the eye, wherein the pincer arms 40 may be utilized to secure the IOL to the iris. The resulting forces produced by haptics 14 in this embodiment are largely contained by the support 22, thus reducing or eliminating the effect of these forces to bend or otherwise deform the optical element 18.

The IOL 10 may be fabricated using various manufacturing methods common in the art. In certain embodiments, a method of manufacturing the IOL 10 comprises providing a foldable material and forming the material to produce the optic 12. The method further comprises forming the material to produce the two haptics 14, such that the thickness tₕ of each haptic 14 is less than or equal to the thickness tₛ of the support 22.

As discussed above herein, the foldable material may comprise silicone, hydrogel, acrylic, hybrid combinations of the same, or any other material providing desirable optical and mechanical properties. The foldable material may be formed by a molding process such as injection molding, compression molding, or cast molding using a disposable mold. Alternatively or additionally, at least a portion of the IOL 10 may be formed using fabrication techniques such as machining or lithography. The surfaces of the optical element 18 may be fabricated to produce either a substantially monofocal or multifocal element. All or portions of the surfaces of the optical element 18 may be substantially conical (e.g., spherical or parabolic), aspherical, or in the form of a diffractive grating. The surfaces may also be non-axisymmetric, for instance with a cylinder component to provide correction for astigmatism.

The present method of manufacturing the IOL 10 is exemplary and is not intended to be limiting. Other manufacturing methods common in the art may be alternatively used to manufacture the IOL 10. For instance, the haptics 14 may be formed separately from the optic 12 and subsequently joined together to form a single piece having a substantially homogeneous material composition throughout.

The IOL may be placed into the eye of a subject using some type of an insertion tool. In certain embodiments, a method of inserting the intraocular lens 10 into an eye comprises providing the IOL 10 and subsequently folding the IOL 10 to form a size and shape suitable for insertion into the eye through a small incision therein. The method further comprises creating an incision in the eye and then inserting the IOL 10 through the incision and into a portion of the eye.

The IOL 10 is generally folded using an insertion tool similar to those currently available in the art. The insertion tool may comprise cartridge with a load chamber for containing the IOL 10 and an insertion tube or cannula for transporting the IOL 10 into the subject's eye. The insertion tool may further comprise a pushrod for advancing the IOL 10 from the load chamber and down the insertion tube or cannula. Typically, the IOL 10 is folded when mounted into the load chamber and/or as the IOL 10 progresses down the insertion tube or cannula. The IOL 10 may be loaded into the load chamber by a practitioner during the insertion procedure into the eye or may alternatively be pre-packaged into the load chamber or other suitable container by, for example, the manufacturer prior to the insertion procedure.

Using embodiments of the IOL 10, the incision in the subject's eye preferably has a dimension of less than about 2.8 mm and even more preferably less than about 2.5 mm. Such a reduced incision size is achieved without substantial detriment, for example, to the optical properties or power of the optical element 18 of the IOL 10. The size and type of incision may depend on the location of the IOL 10 within the eye (e.g., the capsular bag, anterior chamber, or posterior chamber). In the case of a cataract surgery, the use of the IOL 10 may preclude the need for an incision that is larger than that used during the phacoemulsification procedure to remove the natural lens.

The above presents various embodiments of the present invention, including a process of making and using it, in such full, clear, concise, and exact terms as to enable any person skilled in the art to which it pertains to make and use this invention. This invention is, however, susceptible to modifications and alternate constructions from those discussed above which are fully equivalent. Consequently, it is not the intention to limit this invention to the particular embodiments herein disclosed. On the contrary, the intention is to cover all modifications and alternate constructions coming within the spirit and scope of the invention as generally expressed by the following claims, which particularly point out and distinctly claim the subject matter of the invention.

1. An intraocular lens, comprising:
an optic made of a foldable material having:
an optical element with optical power;
a transition region disposed around the entire perimeter of the optical element, the transition region having a thickness of between at least about 0.07 mm and about 0.40 mm; and
a support disposed about at least a portion of the transition region, the thickness of the support being greater than the thickness of the transition region;
at least two haptics integrally formed with the optic and coupled to the support.

2. The intraocular lens of claim 1, wherein the thickness of the support is between at least about 0.25 mm and about 0.60 mm.

3. The intraocular lens of claim 1, wherein the thickness of the transition region is at least about 0.12 mm.

4. The intraocular lens of claim 1, wherein the thickness of the haptics is less than or equal to the thickness of the support.

5. The intraocular lens of claim 1, wherein the material is selected from a group of deformable materials consisting of hydrogel, silicone, acrylic, and hybrid combinations of the same.

6. The intraocular lens of claim 1, wherein the support is disposed around the entire perimeter of the transition region.

7. The intraocular lens of claim 1, wherein each haptic attaches at least at one location on the support.

8. The intraocular lens of claim 1, wherein each haptic attaches at least at two locations on the support.

9. The intraocular lens of claim 1, wherein each haptic forms a substantially planar surface.

10. The intraocular lens of claim 1, wherein each haptic comprises a pair of pincer arms.

11. The intraocular lens of claim 1, wherein the intraocular lens is adapted for insertion into the capsular bag of an eye.

12. The intraocular lens of claim 1, wherein the intraocular lens is adapted for insertion into the anterior chamber of an eye.

13. The intraocular lens of claim 1, wherein the intraocular lens is adapted for insertion into the posterior chamber of an eye.

14. The intraocular lens of claim 1, wherein the optic further comprises a peripheral edge configured to inhibit cell growth on the intraocular lens.

15. An intraocular lens, comprising:
an optic made of a foldable material having:
an optical element with optical power; and
a transition region around the entire perimeter of the optical element, the transition region having a thickness of between at least about 0.07 mm and about 0.40 mm; and
at least two haptics integrally formed with the optic adapted to produce a positioning force when inserted into an eye;
means for isolating the positioning force from the optical element and the transition region.

16. The intraocular lens of claim 15, wherein the thickness of the transition region is at least about 0.12 mm.

17. The intraocular lens of claim 15, wherein the foldable material is selected from a group of deformable materials consisting of hydrogel, silicone, acrylic, and hybrid combinations of the same.

18. The intraocular lens of claim 15, wherein each haptic forms a substantially planar surface.

19. The intraocular lens of claim 15, wherein each haptic comprises a pair of pincer arms.

20. The intraocular lens of claim 15, wherein the intraocular lens is adapted for insertion into the capsular bag of an eye.

21. The intraocular lens of claim 15, wherein the intraocular lens is adapted for insertion into the anterior chamber of an eye.

22. The intraocular lens of claim 15, wherein the intraocular lens is adapted for insertion into the posterior chamber of an eye.

23. The intraocular lens of claim 15, wherein the optic further comprises a peripheral edge configured to inhibit cell growth on the intraocular lens.

24. A method of manufacturing an intraocular lens, comprising:
providing a foldable material;
forming the material to produce an optic having:
an optical element with optical power;
a transition region disposed around the entire perimeter of the optical element, the transition region having a thickness of between at least about 0.07 mm and about 0.40 mm; and
a support disposed about at least a portion of the transition region, the thickness of the support being greater than the thickness of the transition region;
further forming the material to produce at least two haptics, the thickness of the haptics being less than or equal to the thickness of the support.

25. A method of inserting an intraocular lens into an eye, comprising:
providing an intraocular lens having:
an optic made of a foldable material having:
an optical element with optical power;
a transition region disposed around the entire perimeter of the optical element, the transition region having a thickness of between at least about 0.07 mm and about 0.40 mm; and
a support disposed about at least a portion of the transition region, the thickness of the support being greater than the thickness of the transition region;
at least two haptics integrally formed with the optic, the thickness of the haptics being less than or equal to the thickness of the support
folding the intraocular lens;
creating an incision in an eye; and
inserting the intraocular lens through the incision and into a portion of the eye.
